SEQUENCE LISTING (42 sequences)

SEQ ID NO: 1 - protein, 19 aa, Synthetic construct
MGWSCIILFLVATATGVHS

SEQ ID NO: 2 - protein, 121 aa, Synthetic construct
DVKLVESGGGLVKLGGSLKLSCAASGFTFSRYFMSWVRQTPEKRLELVAAINSDGGRTYYPDTVKGRFTISRDNAKNTLYLQMSSLKSEDTALYYCTRHIYSYYEDYFDYWGQGTTLTVSS

SEQ ID NO: 3 - protein, 5 aa, Synthetic construct
RYFMS

SEQ ID NO: 4 - protein, 17 aa, Synthetic construct
AINSDGGRTYYPDTVKG

SEQ ID NO: 5 - protein, 12 aa, Synthetic construct
HIYSYYEDYFDY

SEQ ID NO: 6 - protein, 330 aa, Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 111 aa, Synthetic construct
DVVMTQTPLSLPVSLGDQASISCRSSQSLVHSNGNTYLHWYLQKPGQSPKLLIYKVSKRFSGVPDRFSGSGSGTDFTLKISRVEAEDLGVYFCSQSTHVYTFGGGTKLEIK

SEQ ID NO: 8 - protein, 16 aa, Synthetic construct
RSSQSLVHSNGNTYLH

SEQ ID NO: 9 - protein, 7 aa, Synthetic construct
KVSKRFS

SEQ ID NO: 10 - protein, 8 aa, Synthetic construct
SQSTHVYT

SEQ ID NO: 11 - protein, 107 aa, Synthetic construct [sequence omitted]

SEQ ID NO: 12 - protein, 121 aa, Synthetic construct
QVQLVESGGGVVQPGRSLRLSCAASGFTFSRYFMSWVRQAPGKGLELVAAINSDGGRTYYPASVKGRFTISRDNSKNTLYLQMNSLRAEDTAVYYCTRHIYSYYEDYFDYWGQGTLVTVSS

SEQ ID NO: 13 - protein, 5 aa, Synthetic construct
RYFMS

SEQ ID NO: 14 - protein, 17 aa, Synthetic construct
AINSDGGRTYYPASVKG

SEQ ID NO: 15 - protein, 12 aa, Synthetic construct
HIYSYYEDYFDY

SEQ ID NO: 16 - protein, 330 aa, Synthetic construct; identical to SEQ ID NO: 6

SEQ ID NO: 17 - protein, 111 aa, Synthetic construct
DVVMTQTPLSSPVTLGQPASISCRSSQSLVHSNGNTYLHWYQQRPGQPPKLLIYKVSKRFSGVPDRFSGSGSGTDFTLKISRVEAEDVGVYFCSQSTHVYTFGQGTKLEIK

SEQ ID NO: 18 - protein, 16 aa, Synthetic construct
RSSQSLVHSNGNTYLH

SEQ ID NO: 19 - protein, 7 aa, Synthetic construct
KVSKRFS

SEQ ID NO: 20 - protein, 8 aa, Synthetic construct
SQSTHVYT

SEQ ID NO: 21 - protein, 107 aa, Synthetic construct [sequence omitted]

SEQ ID NO: 22 - protein, 121 aa, Synthetic construct
EVQLVESGGGVVQPGRSLRLSCAASGFTFSRYFMSWVRQAPGKGLELVAAINSDGGRTYYPASVKGRFTISRDNSKNTLYLQMNSLRAEDTAVYYCTRHIYSYYEDYFDYWGQGTLVTVSS

SEQ ID NO: 23 - protein, 5 aa, Synthetic construct
RYFMS

SEQ ID NO: 24 - protein, 17 aa, Synthetic construct
AINSDGGRTYYPASVKG

SEQ ID NO: 25 - protein, 12 aa, Synthetic construct
HIYSYYEDYFDY

SEQ ID NO: 26 - protein, 330 aa, Synthetic construct; identical to SEQ ID NO: 6

SEQ ID NO: 27 - protein, 111 aa, Synthetic construct; identical to SEQ ID NO: 17

SEQ ID NO: 28 - protein, 16 aa, Synthetic construct
RSSQSLVHSNGNTYLH

SEQ ID NO: 29 - protein, 7 aa, Synthetic construct
KVSKRFS

SEQ ID NO: 30 - protein, 8 aa, Synthetic construct
SQSTHVYT

SEQ ID NO: 31 - protein, 107 aa, Synthetic construct [sequence omitted]

SEQ ID NO: 32 - protein, 7 aa, Synthetic construct
KTLGELL

SEQ ID NO: 33 - protein, 22 aa, Synthetic construct
EAPGVRGSTKTLGELLDTGTEL

SEQ ID NO: 34 - protein, 7 aa, Synthetic construct
KTLGELL

SEQ ID NO: 35 - protein, 12 aa, Synthetic construct
RRTCVFFEAPGV

SEQ ID NO: 36 - protein, 9 aa, Synthetic construct
RAIRCLYSR

SEQ ID NO: 37 - DNA, 363 bp, Synthetic construct
  source 1..363: mol_type = other DNA
gacgtgaaactcgtggagtctgggggaggcttagtgaagcttggagggtccctgaaactctcctgtgcagcctctggattcactttcagtaggtatttcatgtcttgggttcgccagactccagagaagaggctggagttggtcgcagccattaatagtgatggtggtaggacctactatccagacactgtgaagggccgattcaccatctccagagacaatgccaagaacaccctgtacctgcaaatgagcagtctgaagtctgaggacacagccttgtattactgtacaagacatatctactcttattacgaggactactttgactactggggccaaggcaccactctcacagtctcctca

SEQ ID NO: 38 - DNA, 333 bp, Synthetic construct
  source 1..333: mol_type = other DNA
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcttgatctacaaagtttccaagcgattttctggggtcccagacaggttcagtggcagtggatcagggacagatttcacactcaagatcagcagagtggaggctgaggatctgggagtttatttctgctctcaaagtacacatgtttacacgttcggaggggggaccaagctggaaataaag

SEQ ID NO: 39 - DNA, 363 bp, Synthetic construct
  source 1..363: mol_type = other DNA
caggtgcagctggtggagtccggcggcggagtggtgcagccaggaagatccctgaggctgagctgcgctgctagcggcttcaccttttccaggtactttatgagctgggtgaggcaggcccctggcaagggcctggagctggtggctgccatcaactccgacggcggccggacctattatcctgctagcgtgaagggccggtttaccatctcccgggacaatagcaagaataccctgtatctgcagatgaatagcctgagggccgaggataccgccgtgtactattgtaccaggcacatctacagctactatgaggattattttgattactggggccagggcaccctggtgaccgtgagcagc

SEQ ID NO: 40 - DNA, 333 bp, Synthetic construct
  source 1..333: mol_type = other DNA
gacgtggtgatgacccagacacccctgtcctcccctgtgacactgggccagcccgcctccatctcctgtagaagcagccagagcctggtgcacagcaacggcaatacctacctccattggtatcagcagagacctggccagcctcccaagctgctgatctacaaggtgagcaagaggttctccggcgtgcccgataggttctccggaagcggcagcggcaccgacttcaccctgaagatcagcagagtggaggccgaggacgtgggcgtgtacttctgtagccagagcacccacgtgtacacattcggccagggcacaaagctggagatcaag

SEQ ID NO: 41 - DNA, 363 bp, Synthetic construct
  source 1..363: mol_type = other DNA
gaggtgcagctggtggagtccggcggcggagtggtgcagccaggaaggtccctgcggctgagctgcgccgctagcggattcactttcagccggtattttatgagctgggtgcggcaggctcctggcaagggcctggagctggtggctgccatcaatagcgacggcggcaggacctattatcctgccagcgtgaagggccggttcaccatctcccgggataactccaagaataccctgtacctccaaatgaatagcctgcgggctgaggataccgccgtgtattattgcacccggcacatctacagctattatgaggactacttcgactactggggccagggcaccctggtgaccgtgtcctcc

SEQ ID NO: 42 - DNA, 333 bp, Synthetic construct; identical to SEQ ID NO: 40
  source 1..333: mol_type = other DNA

(19) United States
(12) Patent Application Publication
(10) Pub. No.: US 2026/0167733 A1
(43) Pub. Date: Jun. 18, 2026

(54) AMHR2-ED BINDING MOLECULES FOR TREATING DISEASES

(71) Applicant: The Cleveland Clinic Foundation, Cleveland, OH (US)

(72) Inventors: Vincent K. TUOHY, Broadview Heights, OH (US); Suparna MAZUMDER, Highland Heights, OH (US); Justin M. JOHNSON, Willoughby Hills, OH (US)

(21) Appl. No.: 19/126,936

(22) PCT Filed: Nov. 2, 2023
(86) PCT No.: PCT/US2023/078469

§ 371 Date: May 2, 2025

Related U.S. Application Data
(60) Provisional application No. 63/382,197, filed on Nov. 3, 2022.

(51) Int. Cl. C07K 16/28; A61K 39/00; A61P 35/00; G01N 33/575; G01N 33/57545; G01N 33/5755; G01N 33/68

(52) U.S. Cl. CPC C07K 16/2869; A61P 35/00; G01N 33/57545; G01N 33/5755; G01N 33/5759; G01N 33/6872; A61K 2039/505; C07K 2317/24; C07K 2317/34; C07K 2317/522; C07K 2317/524; C07K 2317/526; C07K 2317/55; C07K 2317/565; C07K 2317/567; C07K 2317/72; C07K 2317/73; G01N 2333/72

(57) ABSTRACT

Provided herein are extra-cellular domain Mullerian hormone receptor type II (AMHR2-ED) binding molecules and nucleic acid sequences encoding such molecules. In particular embodiments, provided herein are AMHR2-ED binding molecules (e.g., monoclonal antibodies or antigen binding fragments thereof) with particular light and/or heavy chains variable regions (SEQ ID NOs: 12, 17, 22, or 27), or light chain and/or heavy chain CDRs (e.g., selected from SEQ ID NOS: 13, 14, 15, 18, 19, and 20) and methods for using such molecules to treat ovarian and/or endometrial cancers.

The present application claims priority to U.S. Provisional application Ser. No. 63/382,197, filed Nov. 3, 2022, which is herein incorporated by reference in its entirety.

### SEQUENCE LISTING

The instant application contains a Sequence Listing which has been submitted electronically in XML. format and is hereby incorporated by reference in its entirety. Said XML. copy created on Nov. 1, 2023, is named CCF_39708_601_SequenceListing_11-02-2023.xml and is 41,384 bytes.

### FIELD OF THE INVENTION

Provided herein are extra-cellular domain Mullerian hormone receptor type II (AMHR2-ED) binding molecules and nucleic acid sequences encoding such molecules. In particular embodiments, provided herein are AMHR2-ED binding molecules (e.g., monoclonal antibodies or antigen binding fragments thereof) with particular light and/or heavy chains variable regions (SEQ ID NOs: 12, 17, 22, or 27), or light chain and/or heavy chain CDRs (e.g., selected from SEQ ID NOS: 13, 14, 15, 18, 19, and 20) and methods for using such molecules to treat ovarian and/or endometrial cancers.

### BACKGROUND OF THE INVENTION

Epithelial ovarian carcinoma (EOC) is the most prevalent form of ovarian cancer in the USA representing ˜85% of all cases and causing more deaths than any other gynecologic malignancy. Approximately 53% of women are dead within 5 years of diagnosis, and effective treatments are few and often become ineffective due to the frequent and rapid development of drug resistance. Thus, there is an urgent need for more effective treatments for EOC.

### SUMMARY OF THE INVENTION

Provided herein are extra-cellular domain Mullerian hormone receptor type II (AMHR2-ED) binding molecules and nucleic acid sequences encoding such molecules. In particular embodiments, provided herein are AMHR2-ED binding molecules (e.g., monoclonal antibodies or antigen binding fragments thereof) with particular light and/or heavy chains variable regions (SEQ ID NOs: 12, 17, 22, or 27), or light chain and/or heavy chain CDRs (e.g., selected from SEQ ID NOS: 13, 14, 15, 18, 19, and 20) and methods for using such molecules to treat ovarian and/or endometrial cancers.

In some embodiments, provided herein are compositions comprising an AMHR2-ED binding molecule, wherein the AMHR2-ED binding molecule comprises: a) a heavy chain variable region, wherein the heavy chain variable region comprises: i) a CDRH1 amino acid sequence comprising SEQ ID NO:13, or SEQ ID NO:13 one with one or two conservative amino acid changes, ii) a CDRH2 amino acid sequence comprising SEQ ID NO:14, and iii) a CDRH3 amino acid sequence comprising SEQ ID NO:15, or SEQ ID NO:15 with one or two conservative amino acid changes, and optionally (in some embodiments), b) a light chain variable region, wherein the light chain variable region comprises; i) a CDRL1 amino acid sequence comprising SEQ ID NO:18, or SEQ ID NO:18 with one or two conservative amino acid changes, ii) a CDRL2 amino acid sequence comprising SEQ ID NO:19, or SEQ ID NO: 19 with one or two conservative amino acid changes, and iii) a CDRL3 amino acid sequence comprising SEQ ID NO:20, or SEQ ID NO:20 with one or two conservative amino acid changes.

In other embodiments, provided herein are compositions comprising: at least one of the following: a) a first nucleic acid sequence encoding a heavy chain variable region, wherein the heavy chain variable region comprises: i) a CDRH1 amino acid sequence comprising SEQ ID NO:13, or SEQ ID NO:13 one with one or two conservative amino acid changes, ii) a CDRH2 amino acid sequence comprising SEQ ID NO:14, and iii) a CDRH3 amino acid sequence comprising SEQ ID NO:15, or SEQ ID NO:15 with one or two conservative amino acid changes, and optionally (in some embodiments), b) a second nucleic acid sequence encoding a light chain variable region, wherein the light chain variable region comprises: i) a CDRL1 amino acid sequence comprising SEQ ID NO:18, or SEQ ID NO:18 with one or two conservative amino acid changes, ii) a CDRL2 amino acid sequence comprising SEQ ID NO:19, or SEQ ID NO:19 with one or two conservative amino acid changes, and iii) a CDRL3 amino acid sequence comprising SEQ ID NO:20, or SEQ ID NO: 20 with one or two conservative amino acid changes. In other embodiments, the compositions further comprise an expression vector, and wherein the first and/or second nucleic acid sequences are present in the expression vector.

In additional embodiments, the AMHR2-ED binding molecule is an antibody, minibody, diabody, scFv, or antibody fragment capable of binding human AMHR2-ED. In further embodiments, the antibody fragment is a Fab, or Fv antibody fragment. In some embodiments, the antibody or antibody fragment comprises an antigen binding portion of the 4D12G1 Variant 5 antibody, or 4D12G1 Variant 6 antibody.

In certain embodiments, the heavy chain and/or light chain variable region comprises a human framework region. In other embodiments, the AMHR2-ED binding molecule further comprises a light chain constant region and a CH1 heavy chain constant region. In particular embodiments, the AMHR2-ED binding molecule further comprises a CH2 heavy chain constant region and/or a CH3 heavy chain constant region. In certain embodiments, the light chain constant region is human or a humanized murine, and/or wherein the CH1, CH2, and CH3 heavy chain constant regions are human or are humanized murine. In additional embodiments, the AMHR2-ED binding molecule is an antibody or antigen binding portion thereof that has an Fc region characterized in that it: i) has an Fc cellular binding site; ii) has a Fc complement binding site; and/or 3) has a linked toxin that is internalized into a cell.

In some embodiments, the AMHR2-ED binding molecule comprises an antibody, wherein the light chain constant region of the antibody is selected from: IgG Kappa and IgG Lambda, and wherein the heavy chain constant region of the antibody is selected from: IgG1, IgG2, IgG3, and IgG4. In further embodiments, the AMHR2-ED binding molecule comprises an antibody, or antigen binding portion thereof, which is glycosylated or non-glycosylated. In additional embodiments, the compositions further comprise: a physiologically tolerable buffer.

In certain embodiments, the heavy chain variable regions comprises SEQ ID NO:12 or 22, or SEQ ID NOs: 12 or 22 with one or more conservative amino acid changes. In further embodiments, the light chain variable region comprises SEQ ID NO: 17 or 27, or SEQ ID NOs: 17 or 27 with one or more conservative amino acid changes.

In some embodiments, provided herein are methods of treating or preventing ovarian and/or endometrial cancer comprising: treating a subject with an AMHR2-ED binding molecule as recited above and herein, or an expression vector encoding the AMHR2-ED binding molecules above and herein, and wherein the subject has, or is suspected to develop, ovarian and/or endometrial cancer. In certain embodiments, the ovarian and/or endometrial cancer is ovarian cancer. In further embodiments, the ovarian and/or endometrial cancer is endometrial cancer.

In certain embodiments, the AMHR2-ED binding molecule is an antibody or antigen binding portion thereof. In other embodiments, the antibody or antigen binding portion thereof is a human antibody or antigen binding portion thereof. In additional embodiments, the antibody or antigen binding portion thereof is a humanized antibody or antigen binding portion thereof. In certain embodiments, the heavy chain variable regions comprises SEQ ID NO: 12 or 22, or SEQ ID NOs: 12, or 22 with one or more conservative amino acid changes. In some embodiments, the light chain variable region comprises SEQ ID NO:17 or 27, or SEQ ID NOs: 17 or 27 with one or more conservative amino acid changes.

In particular embodiments, provided herein are methods of detecting AMHR2-ED in a sample comprising: a) contacting a sample with the AMHR2-ED binding molecule described above or herein, wherein the sample is suspected of containing AMHR2-ED, and wherein the AMHR2-ED binding molecule forms a complex with the AMHR2-ED if present in the sample; and b) detecting the presence or absence of the complex in the sample. In particular embodiments, the sample is from a subject that has, or is suspected to develop, ovarian and/or endometrial cancer. In other embodiments, the AMHR2-ED binding molecule comprises a detectable label. In additional embodiments, the methods further comprise: contacting the sample with a conjugate molecule capable of binding to the AMHR2-ED binding molecule, wherein the conjugate molecule comprises a detectable label.

### DESCRIPTION OF THE FIGURES

FIG. 1 shows a schematic representation of an exemplary IgG molecule with the various regions and sections labeled. The CDRs and framework regions (FR) of one of the two variable region light chains, and one of the two variable region heavy chains, are also labeled.

FIG. 2 (A and B) shows the percent binding of various mAbs to EOC Cells, showing that the Chimeric 4D12G1 mAb and its humanized variants 5 and 6 bind effectively to human EOC cells expressing AMHR2-ED. (C) Variant 6 was used in immunohistochemical staining (20×) of tumor tissue sections from three HGSOC patients (#1-3). Staining was predominantly in the tumor parenchyma with some staining occurring in the stromal areas of the tumors. Corner insets show detail at 40× from the corresponding areas framed by smaller rectangles. All error bars indicate ±SD.

FIG. 3 shows that chimeric and humanized 4D12G1 mAbs induce programmed cell death (PCD) determined by MTS (A) and IncuCyte Caspase 3/7 (B) assays. Specifically, FIG. 3 shows the chimeric 4D12G1 mAb and its humanized variants 5 and 6 induce PCD of human EOC cells expressing AMHR2-ED. All error bars indicate ±SD.

FIG. 4 shows epitope mapping of the humanized 4D12G1 variants 5 and 6 mAbs in binding ELISAs to AMHR2-ED peptides. An overlapping series of 16-mer peptides spanning the entire sequence of human AMHR2-ED with one amino acid shift was plated for direct ELISA testing using either Variant 5 or Variant 6 as the primary antibody. Optical densities of the color reactions were determined by absorbance at 405 nm and plotted as a Y-axis. Both variants recognized residues AMHR2-ED 11-33, the same residues recognized by mouse 4D12G1 mAb. All error bars indicate ±SD.

FIG. 5 shows in vivo inhibition of human EOC growth by humanized mAbs. Specifically, FIG. 75 shows the 4D12G1 chimeric and variant 6 mAbs, but not the 4D12G1 variant 5 mAb, inhibited the growth of human AMHR2-OVCAR8 EOCs inoculated into immunodeficient NSG mice. All error bars indicate ±SD and asterisks indicate significance (P<0.0001 for both chimeric and Variant 6 mAbs and P=0.003 for Variant 5 mAb compared with control).

FIG. 6 shows the structural comparison of mouse 4D12G1, Chimeric 4D12G1, and humanized variant 6 (Hu4D12G1-6). Molecular modeling of the monoclonal antibodies was performed using BioLuminate software and alignments were done using the Protein Structure Alignment algorithm. Blue hues represent heavy chains, and red hues represent light chains. (A) Complementarity determining regions (CDRs) for the variable heavy chain (VH; H1-H3) and the variable kappa light chain (VK; L1-L3) are indicated for mouse 4D12G1. (B) Chimeric 4D12G1 consists of the variable heavy and light chains of mouse 4D12G1 fused to the constant heavy and light chains of human IgG1, respectively. (C) Hu4D12G1-6 of the humanized version of 4D12G1 Fab is shown superimposed on the parental mouse 4D12G1 Fab. The key antigen-binding residues Ala50, Ser35, and Leu47 in the variable heavy chain are represented as balls and sticks and are indicated by magenta, purple, and green arrows, respectively. (D) The conserved key residues in the superimposed model are shown with greater magnification for clarity.

FIG. 7 shows that Hu4D12G1-6 induces apoptosis in vitro. (A) AMHR2-OVCAR8 cells were treated with 25 μg/ml Hu4D12G1-6 or with isotype control to cultures in the CaspaTag Caspase-3/7 assay and indicated substantial Caspase-3/7 activity in the Hu4D12G1-6 treated group versus the control (P<0.0001). (B) Western blot analysis of cleaved Caspase-3 proteins following treatment with Hu4D12G1-6 (5-20 μg/ml) for 16 hours of AMHR2-OVCAR8 cells facilitated the generation of cleaved Caspase-3. Immunostaining with β-actin antibody was used to confirm normalized lysate loading. (C) Apoptosis of AMHR2-OVCAR8 was imaged in real-time using Incucyte S3 live-cell analysis with green fluorescent active Caspase-3/7 dye, suggesting a significant number of Caspase-3/7 positive cells induced with Hu4D12G1-6 treatment compared to the isotype control. (D) The live-cell analysis data were plotted as percent Caspase-3 green fluorescent dye positive cells treated with 10 μg/ml Hu4D12G1-6 for 16 hours induced significant Caspase-3/7 activation compared to cultures treated with isotype control (˜40%, P=0.0007). The results shown in (A), (B), (C), and (D) indicate that Hu4D12G1-6 treatment induces significant programmed cell death in AMHR2-OVCAR8 cells. All error bars indicate ±SD.

FIG. 8 shows that Hu4D12G1-6 inhibits growth of human EOC xenografts in mice. Human EOC tumors were injected s.c. into immunodeficient NSG, humanized NSG-SGM3, and humanized NSG-IL-15 mice. When tumors became palpable, mice were injected i.p. with 200 μg of either Hu4D12G1-6 or an isotype control weekly for 5 consecutive weeks. Treatment with Hu4D12G1-6 significantly inhibited the growth of (A) AMHR2-OVCAR8 tumors (P<0.0001), and (B) primary HGSOC tumor cell lines generated from a patient, PDX #10 (P=0.0005) in severely immunodeficient NSG mice. Additionally, treatment with Hu4D12G1-6 significantly inhibited the growth of AMHR2-OVCAR8 tumors in (C) huNSG-IL-15 mice (P<0.0001), and in (D) huNSG-SGM3 mice (P<0.0001). Taken together, these data indicate that Hu4D12G1-6 is efficacious for inhibiting human EOCs xenografts in mice. All error bars indicate ±SD.

FIG. 9 shows that Hu4D12G1-6 inhibits EOC predominantly by inducing apoptosis. (A) Caspase-3 IHC of AMHR2-OVCAR8 derived tumors in NSG mice were examined and showed pronounced Caspase-3 positive cells in the tumor beds of Hu4D12G1-6 treated mice, indicating programmed cell death. Similarly, Caspase-3 IHC (left side) of (B) PDX #10 and (C) AMHR2-OVACR8 derived tumors in huNSG-SGM3 mice indicate that apoptosis is the predominant phenomenon in the tumor bed with Hu4D12G1-6 treatment. Corner insets show detail at 40× from the corresponding areas of 20× framed by smaller rectangles. The quantification of Caspase-3 (A), (B), and (C) positive cells per section were plotted on the right side of the corresponding IHC FIGS. 4a, P=0.0019; 6b, P=0.0034; 6c, P=0.0451). All error bars indicate ±SD.

FIG. 10 shows the signal peptide employed and the amino acid sequence of the Anti-AMHR2-ED 4D12G1 Chimeric antibody. FIG. 10A shows the amino acid sequence of the signal peptide employed (SEQ ID NO:1). FIG. 10B shows the amino acid sequence of the heavy chain variable region (SEQ ID NO:2), including CDRH1 (SEQ ID NO:3), CDRH2 (SEQ ID NO:4), and CDRH3 (SEQ ID NO:5). FIG. 10C shows the amino acid sequence of the heavy chain constant regions (SEQ ID NO:6). FIG. 10D shows the amino acid sequence of the signal peptide employed (SEQ ID NO:1). FIG. 10E shows the amino acid sequence of the light chain variable region (SEQ ID NO:7), including CDRL1 (SEQ ID NO: 8), CDRL2 (SEQ ID NO:9), and CDRL3 (SEQ ID NO:10). FIG. 10F shows the amino acid sequence of the light chain constant region (SEQ ID NO:11). Kabat definitions of CDRs were employed.

FIG. 11 shows the signal peptide employed and the amino acid sequence of the Anti-AMHR2-ED 4D12G1 Variant 5 antibody. FIG. 11A shows the amino acid sequence of the signal peptide employed (SEQ ID NO:1). FIG. 11B shows the amino acid sequence of the heavy chain variable region (SEQ ID NO: 12), including CDRH1 (SEQ ID NO:13), CDRH2 (SEQ ID NO:14), and CDRH3 (SEQ ID NO:15). FIG. 11C shows the amino acid sequence of the heavy chain constant regions (SEQ ID NO:16). FIG. 11D shows the amino acid sequence of the signal peptide employed (SEQ ID NO:1). FIG. 11E shows the amino acid sequence of the light chain variable region (SEQ ID NO:17), including CDRL1 (SEQ ID NO: 18), CDRL2 (SEQ ID NO:19), and CDRL3 (SEQ ID NO:20). FIG. 11F shows the amino acid sequence of the light chain constant region (SEQ ID NO:21). Kabat definitions of CDRs were employed.

FIG. 12 shows the signal peptide employed and the amino acid sequence of the Anti-AMHR2-ED 4D12G1 Variant 6 antibody. FIG. 12A shows the amino acid sequence of the signal peptide employed (SEQ ID NO:1). FIG. 12B shows the amino acid sequence of the heavy chain variable region (SEQ ID NO:22), including CDRH1 (SEQ ID NO:23), CDRH2 (SEQ ID NO:24), and CDRH3 (SEQ ID NO:25). FIG. 12C shows the amino acid sequence of the heavy chain constant regions (SEQ ID NO:26). FIG. 12D shows the amino acid sequence of the signal peptide employed (SEQ ID NO:1). FIG. 12E shows the amino acid sequence of the light chain variable region (SEQ ID NO:27), including CDRL1 (SEQ ID NO: 28), CDRL2 (SEQ ID NO:29), and CDRL3 (SEQ ID NO:30). FIG. 12F shows the amino acid sequence of the light chain constant region (SEQ ID NO:31). Kabat definitions of CDRs were employed.

FIG. 13 shows nucleic acid sequences encoding of the Anti-AMHR2-ED 4D12G1 Chimeric antibody, Variant 5, and Variant 6, as follows: A) Chimeric 4D12G1 VH DNA sequence (SEQ ID NO:37); B) Chimeric 4D12G1 VK DNA sequence (SEQ ID NO:38); C) Variant 5 (HU) VH (SEQ ID NO:39); D) Variant 5 (KB) VK (SEQ ID NO:40); E) Variant 6 (HV) VH (SEQ ID NO:41); and F) Variant 6 (KB) VK (SEQ ID NO:42).

### DEFINITIONS

To facilitate an understanding of the invention, a number of terms are defined below.

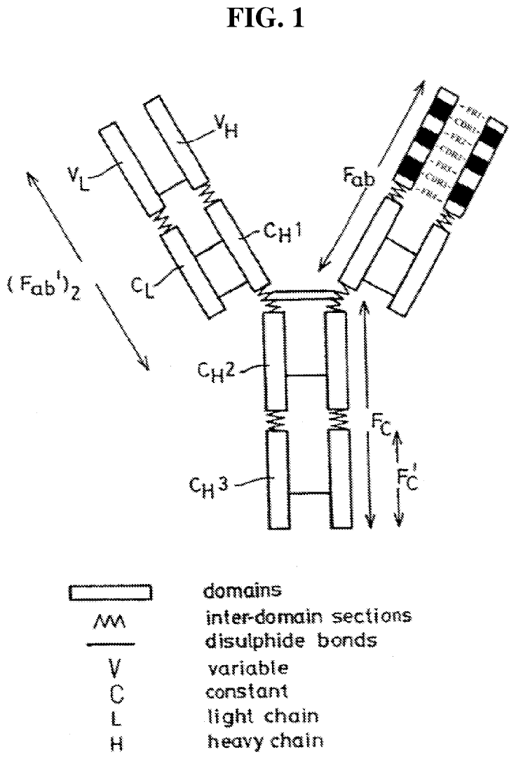

The term “antibody,” as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each variable region (VH or VL) contains 3 CDRs, designated CDR1, CDR2 and CDR3 (see, FIG. 1). Each variable region also contains 4 framework sub-regions, designated FR1, FR2, FR3 and FR4 (see, FIG. 1), which may be human framework sub-regions.

As used herein, the term “antibody fragment or portion” refers to a portion of an intact antibody. Examples of antibody fragments or portions include, but are not limited to, linear antibodies, single-chain antibody molecules, Fv, Fab and F(ab′) 2 fragments, and multi-specific antibodies formed from antibody fragments. The antibody fragments preferably retain at least part of the heavy and/or light chain variable region.

As used herein, the terms “complementarity determining region” and “CDR” refer to the regions that are primarily responsible for antigen-binding. There are three CDRs in a light chain variable region (CDRL1, CDRL2, and CDRL3), and three CDRs in a heavy chain variable region (CDRH1, CDRH2, and CDRH3).

As used herein, the term “framework” refers to the residues of the variable region other than the CDR residues. There are four separate framework sub-regions that make up the framework: FR1, FR2, FR3, and FR4 (see, FIG. 1). In order to indicate if the framework sub-region is in the light or heavy chain variable region, an “L” or “H” may be added to the sub-region abbreviation (e.g., “FRL1” indicates framework sub-region 1 of the light chain variable region). It is noted that, in certain embodiments, the AMHR2-ED binding molecules of the present invention may have less than a complete framework (e.g. the AMHR2-ED binding molecule may have a portion of a framework that only contains one or more of the four sub-regions).

As used herein, the term “fully human framework” means a framework with an amino acid sequence found naturally in humans. Examples of fully human frameworks, include, but are not limited to, KOL, NEWM, REI, EU, TUR, TEI, LAY and POM (See, e.g., Kabat et al., (1991) Sequences of Proteins of Immunological Interest, US Department of Health and Human Services, NIH, USA; and Wu et al., (1970) J. Exp. Med. 132, 211-250, both of which are herein incorporated by reference).

As used herein, the terms “subject” and “patient” refer to any animal, such as a mammal like a dog, cat, bird, livestock, and preferably a human.

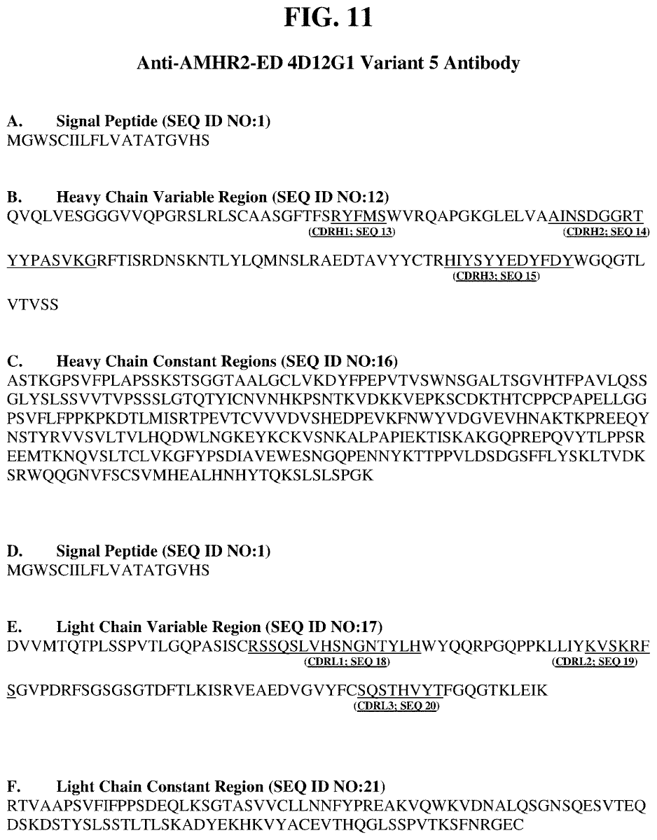

As used herein, the term “codon” or “triplet” refers to a group of three adjacent nucleotide monomers which specify one of the naturally occurring amino acids found in polypeptides. The term also includes codons which do not specify any amino acid. It is also noted that, due to the degeneracy of the genetic code, there are many codons that code for the same amino acid. As such, many of the bases of the nucleic acid sequences of the present invention (see, FIG. 11, or underlined CDRs therein) can be changed without changing the actual amino acid sequence that is encoded. The present disclosure is intended to encompass all such nucleic acid sequences.

As used herein, the terms “an oligonucleotide having a nucleotide sequence encoding a polypeptide,” “polynucleotide having a nucleotide sequence encoding a polypeptide,” and “nucleic acid sequence encoding a peptide” means a nucleic acid sequence comprising the coding region of a particular polypeptide. The coding region may be present in a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc., or a combination of both endogenous and exogenous control elements.

Also, as used herein, there is no size limit or size distinction between the terms “oligonucleotide” and “polynucleotide.” Both terms simply refer to molecules composed of nucleotides. Likewise, there is no size distinction between the terms “peptide” and “polypeptide.” Both terms simply refer to molecules composed of amino acid residues.

As used herein, the term “the complement of” a given sequence is used in reference to the sequence that is completely complementary to the sequence over its entire length. For example, the sequence 5′-A-G-T-A-3′ is “the complement” of the sequence 3′-T-C-A-T-5′. The present disclosure also provides the complement of the sequences described herein (e.g., the complement of the nucleic acid sequences shown in FIG. 11, and truncated versions thereof).

The term “isolated” when used in relation to a nucleic acid, as in “an isolated oligonucleotide” or “isolated polynucleotide” or “isolated nucleic acid sequence encoding an AMHR2-ED binding molecule” (see, e.g., FIG. 15) refers to a nucleic acid sequence that is identified and separated from at least one contaminant nucleic acid with which it is ordinarily associated (e.g. host cell proteins).

As used herein, the term “purified” or “to purify” refers to the removal of contaminants from a sample. For example, AMHR2-ED binding molecules (e.g., antibodies or antibody fragments) may be purified by removal of contaminating non-immunoglobulin proteins; they are also purified by the removal of immunoglobulins that do not bind to the same antigen. The removal of non-immunoglobulin proteins and/or the removal of immunoglobulins that do not bind the particular antigen results in an increase in the percentage of antigen specific immunoglobulins in the sample. In another example, recombinant antigen-specific polypeptides are expressed in bacterial host cells and the polypeptides are purified by the removal of host cell proteins; the percentage of recombinant antigen-specific polypeptides is thereby increased in the sample.

As used herein, the term “Fc region” refers to a C-terminal region of an immunoglobulin heavy chain. The “Fc region” may be a native sequence Fc region or a variant Fc region (e.g., with increased or decreased effector functions).

### DESCRIPTION OF THE INVENTION

Provided herein are extra-cellular domain Mullerian hormone receptor type II (AMHR2-ED) binding molecules and nucleic acid sequences encoding such molecules. In particular embodiments, provided herein are AMHR2-ED binding molecules (e.g., monoclonal antibodies or antigen binding fragments thereof) with particular light and/or heavy chains variable regions (SEQ ID NOs: 12, 17, 22, or 27), or light chain and/or heavy chain CDRs (e.g., selected from SEQ ID NOS: 13, 14, 15, 18, 19, and 20) and methods for using such molecules to treat ovarian and/or endometrial cancers.

In certain embodiments, provided herein are the 4D12G1 chimeric mAb and its two humanized 4D12G1 variant mAbs (variants 5 and 6) which show binding and immunologic features similar to the native mouse 4D12G1 mAb from which they were derived including similar binding to human EOC cells expressing AMHR2-ED, the ability to induce death of human EOC cells by programmed cell death, and recognition of the same important AMHR2-ED 20-26 sequence (20KTLGELL26, SEQ ID NO:32) recognized by the native mouse 4D12G1 mAb. In work conducted during the development of embodiments herein, the humanized 4D12G1 variant 6 mAb, but not the 4D12G1 Variant 5 mAb, inhibited the growth of human EOC tumors growing in immunodeficient NSG mice after inoculated with AMHR2-OVCAR8 EOC cells.

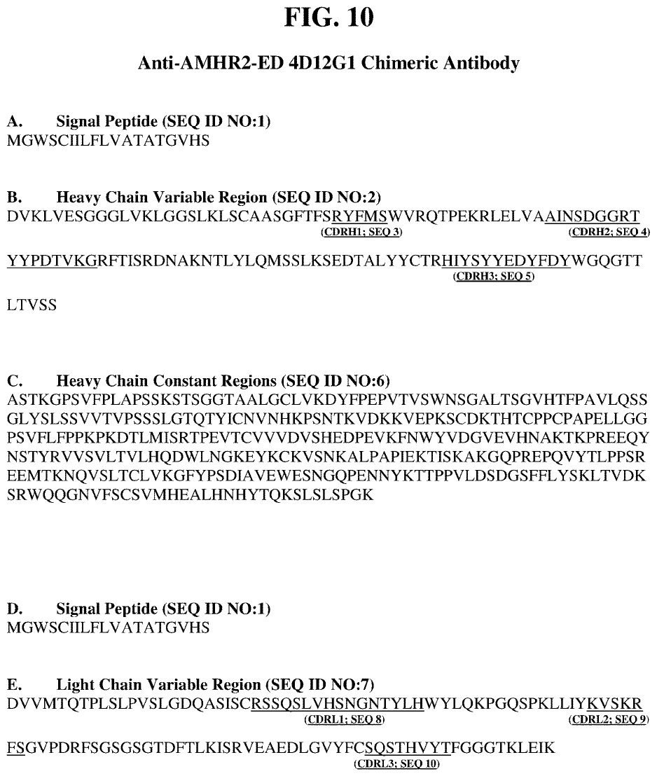

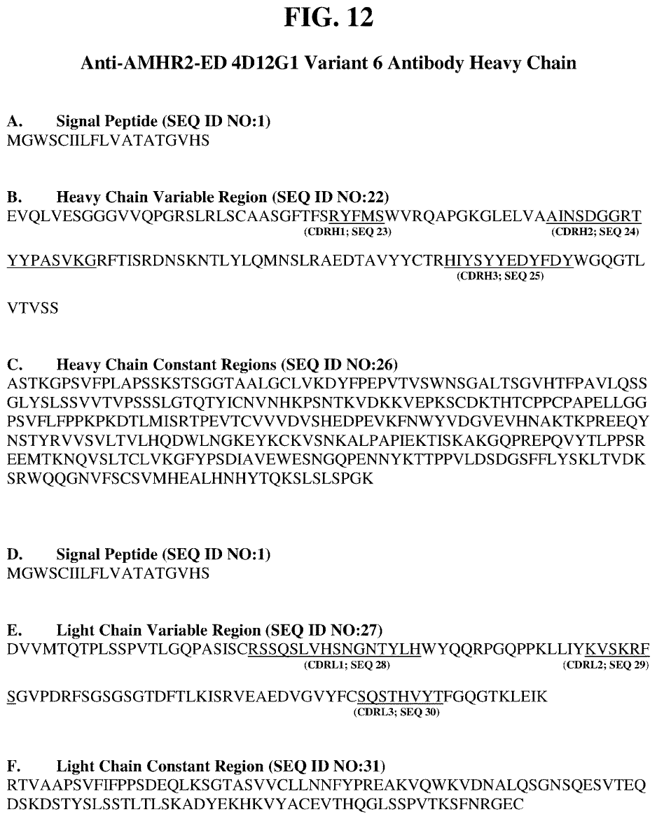

In certain embodiments, the AMHR2-ED binding molecules comprise one or more of the CDRs shown in SEQ ID NOS: 13, 14, 15, 18, 19, and 20 and/or CDRs with one or more conservative or non-conservative amino acid changes in these SEQ ID NOS. Also provided are nucleic acid sequences substantially similar to SEQ ID NOS: 37-42 (e.g., sequences with at least 80 . . . 90 . . . 95% . . . or 99% sequence identity). Changes to the amino acid sequences of the CDRs or variable regions (see FIGS. 10-12) may be generated by changing the nucleic acid sequence encoding the amino acid sequence. A nucleic acid sequence encoding a variant of a given CDR or variable region may be prepared by methods known in the art using the guidance of the present specification for particular sequences. These methods include, but are not limited to, preparation by site-directed (or oligonucleotide-mediated) mutagenesis, PCR mutagenesis, and cassette mutagenesis of an earlier prepared nucleic acid encoding the CDR or variable region.

Briefly, in carrying out site-directed mutagenesis of DNA, the starting DNA is altered by first hybridizing an oligonucleotide encoding the desired mutation to a single strand of such starting DNA. After hybridization, a DNA polymerase is used to synthesize an entire second strand, using the hybridized oligonucleotide as a primer, and using the single strand of the starting DNA as a template. Thus, the oligonucleotide encoding the desired mutation is incorporated in the resulting double-stranded DNA.

PCR mutagenesis is also suitable for making amino acid sequence variants of the starting CDR (see, e.g., Vallette et. al., (1989) Nucleic Acids Res. 17:723-733, hereby incorporated by reference). Briefly, when small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template.

Another method for preparing variants, cassette mutagenesis, is based on the technique described by Wells et al., (1985) Gene 34:315-323, hereby incorporated by reference. The starting material is the plasmid (or other vector) comprising the starting CDR or variant region DNA to be mutated. The codon(s) in the starting DNA to be mutated are identified. There should be a unique restriction endonuclease site on each side of the identified mutation site(s). If no such restriction sites exist, they may be generated using the above-described oligonucleotide-mediated mutagenesis method to introduce them at appropriate locations in the starting polypeptide DNA. The plasmid DNA is cut at these sites to linearize it. A double-stranded oligonucleotide encoding the sequence of the DNA between the restriction sites but containing the desired mutation(s) is synthesized using standard procedures, wherein the two strands of the oligonucleotide are synthesized separately and then hybridized together using standard techniques. This double-stranded oligonucleotide is referred to as the cassette. This cassette is designed to have 5′ and 3′ ends that are compatible with the ends of the linearized plasmid, such that it can be directly ligated to the plasmid. This plasmid now contains the mutated DNA sequence.

Alternatively, or additionally, the desired amino acid sequence encoding a CDR variant, or variable region variant, can be determined, and a nucleic acid sequence encoding such amino acid sequence variant can be generated synthetically. Conservative modifications in the amino acid sequences of the CDRs or variable region may also be made. Naturally occurring residues are divided into classes based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.
   Conservative substitutions will entail exchanging a member of one of these classes for another member of the same class in a particular CDR, such as in SEQ ID NOS: 13, 14, 15, 18, 19, and 20. The present invention also provides the complement of the nucleic acid sequences shown SEQ ID NOS: 37-42, as well as nucleic acid sequences that will hybridize to these nucleic acid sequences under low, medium, and high stringency conditions.

The CDRs of the present invention may be employed with any type of suitable framework. In some embodiments, the CDRs are used with fully human frameworks, or framework sub-regions. For example, the NCBI web site contains the sequences for known human framework regions. Examples of human VH sequences include, but are not limited to, VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81, which are provided in Matsuda et al., (1998) J. Exp. Med. 188:1973-1975, that includes the complete nucleotide sequence of the human immunoglobulin chain variable region locus, herein incorporated by reference. Examples of human VK sequences include, but are not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8, which are provided in Kawasaki et al., (2001) Eur. J. Immunol. 31:1017-1028; Schable and Zachau, (1993) Biol. Chem. Hoppe Seyler 374:1001-1022; and Brensing-Kuppers et al., (1997) Gene 191:173-181, all of which are herein incorporated by reference. Examples of human VL sequences include, but are not limited to, V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6, which are provided in Kawasaki et al., (1997) Genome Res. 7:250-261, herein incorporated by reference. Fully human frameworks can be selected from any of these functional germline genes. Generally, these frameworks differ from each other by a limited number of amino acid changes. These frameworks may be used with the CDRs described herein. Additional examples of human frameworks which may be used with the CDRs of the present invention include, but are not limited to, KOL, NEWM, REI, EU, TUR, TEI, LAY and POM (See, e.g., Kabat et al., (1991) Sequences of Proteins of Immunological Interest, US Department of Health and Human Services, NIH, USA; and Wu et al., (1970), J. Exp. Med. 132:211-250, both of which are herein incorporated by reference).

In certain embodiments, the AMHR2-ED binding molecules of the present invention comprise antibodies or antibody fragments (e.g., comprising one or more of the CDRs described herein). An antibody, or antibody fragment, of the present invention can be prepared, for example, by recombinant expression of immunoglobulin light and heavy chain genes in a host cell. For example, to express an antibody recombinantly, a host cell may be transfected with one or more recombinant expression vectors carrying DNA fragments encoding the immunoglobulin light and heavy chains of the antibody such that the light and heavy chains are expressed in the host cell and, preferably, secreted into the medium in which the host cell is cultured, from which medium the antibody can be recovered. Standard recombinant DNA methodologies may be used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al., all of which are herein incorporated by reference.

In certain antibodies, the anti-AMHR2-ED antibodies, or fragments, thereof prepared herein have an IgG isotype constant regions as shown in Table 1 below.

TABLE 1

| IgG Isotype | |
|---|---|
| Constant region | Accession # |
| IgG Light Kappa | P0DOX7.1 |
| IgG Light Lambda | P0DOX8.1 |
| IgG1 Heavy | P01857 |
| IgG2 Heavy | P01859 |
| IgG3 Heavy | P01860 |
| IgG4 Heavy | P01861 |

To express an antibody with one or more of the CDRs herein, DNA fragments encoding the light and heavy chain variable regions are first obtained. These DNAs can be obtained by amplification and modification of germline light and heavy chain variable sequences using the polymerase chain reaction (PCR).

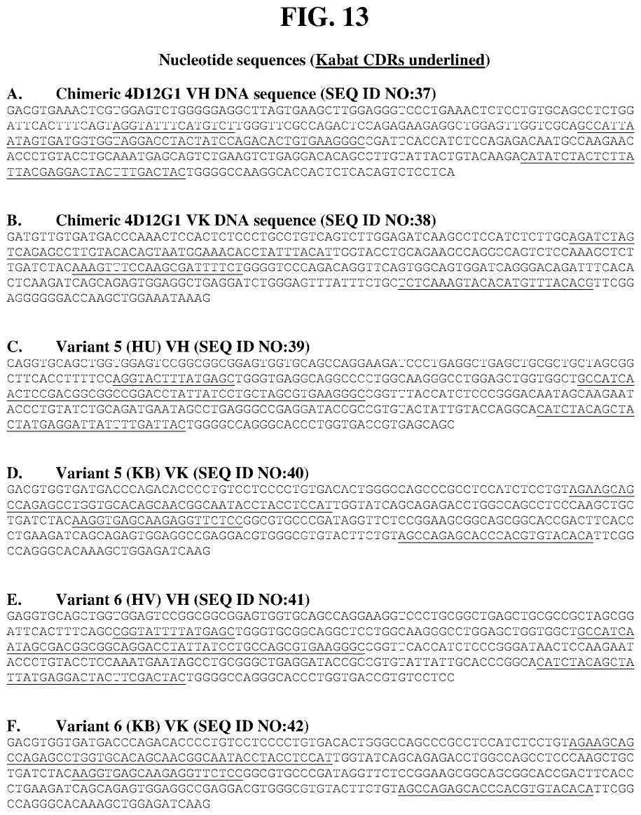

Once the germline VH and VL fragments are obtained, these sequences can be mutated to encode one or more of the CDR amino acid sequences disclosed herein (see, e.g., SEQ ID NOS: 13, 14, 15, 18, 19, or 20). The amino acid sequences encoded by the germline VH and VL DNA sequences may be compared to the CDRs sequence(s) desired to identify amino acid residues that differ from the germline sequences. Then the appropriate nucleotides of the germline DNA sequences are mutated such that the mutated germline sequence encodes the selected CDRs (e.g., the six CDRs shown in FIGS. 10-13), using the genetic code to determine which nucleotide changes should be made. Mutagenesis of the germline sequences may be carried out by standard methods, such as PCR-mediated mutagenesis (in which the mutated nucleotides are incorporated into the PCR primers such that the PCR product contains the mutations) or site-directed mutagenesis. In other embodiments, the variable region is synthesized de novo (e.g., using a nucleic acid synthesizer).

Once DNA fragments encoding the desired VH and VL segments are obtained (e.g., by amplification and mutagenesis of germline VH and VL genes, or synthetic synthesis, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operably linked to another DNA fragment encoding another polypeptide, such as an antibody constant region or a flexible linker. The isolated DNA encoding the VH region can be converted to a full-length heavy chain gene by operably linking the VH-encoding DNA to another DNA molecule encoding heavy chain constant regions (CH1, CH2 and CH3). The sequences of mouse and human heavy chain constant region genes are known in the art and DNA fragments encompassing these regions can be obtained by standard PCR amplification. The heavy chain constant region can be, for example, an IgG1, IgG2, IgG3, IgG4, IgA, IgE, IgM or IgD constant region. For a Fab fragment heavy chain gene, the VH-encoding DNA can be operably linked to another DNA molecule encoding only the heavy chain CH1 constant region.

The isolated DNA encoding the VL region can be converted to a full-length light chain gene (as well as a Fab light chain gene) by operably linking the VL-encoding DNA to another DNA molecule encoding the light chain constant region, CL. The sequences of mouse and human light chain constant region genes are known in the art (see e.g., Kabat, E. A., et al., (1991) Sequences of Proteins of immunological Interest, Fifth Edition, U.S. Department of Health and Human Services. NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments may be operably linked to another fragment encoding a flexible linker, e.g., encoding the amino acid sequence (Gly4-Ser) 3, such that the VH and VL sequences can be expressed as a contiguous single-chain protein, with the VL and VH regions joined by the flexible linker (see e.g., Huston et al., (1988) Proc. Natl. Acad. Sci. USA 85:5879-5883; and McCafferty et al., (1990) Nature 348:552-554), all of which are herein incorporated by reference).

To express the antibodies, or antibody fragments of the invention, DNAs encoding partial or full-length light and heavy chains, (e.g. obtained as described above), may be inserted into expression vectors such that the genes are operably linked to transcriptional and translational control sequences. In this context, the term “operably linked” is intended to mean that an antibody gene is ligated into a vector such that transcriptional and translational control sequences within the vector serve their intended function of regulating the transcription and translation of the antibody gene. The expression vector and expression control sequences are generally chosen to be compatible with the expression host cell used. The antibody light chain gene and the antibody heavy chain gene can be inserted into separate vectors or, more typically, both genes are inserted into the same expression vector. The antibody genes may be inserted into the expression vector by standard methods (e.g., ligation of complementary restriction sites on the antibody gene fragment and vector, or blunt end ligation if no restriction sites are present). Prior to insertion of the light or heavy chain sequences, the expression vector may already carry antibody constant region sequences. For example, one approach to converting the VH and VL sequences to full-length antibody genes is to insert them into expression vectors already encoding heavy chain constant and light chain constant regions, respectively, such that the VH segment is operably linked to the CH segment(s) within the vector and the VL segment is operably linked to the CL segment within the vector. Additionally, or alternatively, the recombinant expression vector can encode a signal peptide that facilitates secretion of the antibody chain from a host cell (e.g., SEQ ID NO: 1, shown in FIG. 10). The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the disclosure may carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990), herein incorporated by reference. It will be appreciated by those skilled in the art that the design of the expression vector, including the selection of regulatory sequences may depend on such factors as the choice of the host cell to be transformed, the level of expression of protein desired, etc. In certain embodiments, regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma virus. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al., all of which are herein incorporated by reference.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neomycin gene (for G418 selection).

For expression of the light and heavy chains, the expression vector(s) encoding the heavy and light chains may be transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

In certain embodiments, the expression vector used to express the AMHR2-ED binding molecules of the present invention are viral vectors, such as retro-viral vectors. Such viral vectors may be employed to generate stably transduced cell lines (e.g. for a continues source of the AMHR2-ED binding molecules). In some embodiments, the GPEX gene product expression technology (from Catalent, Somerset, NJ) is employed to generate AMHR2-ED binding molecules (and stable cell lines expressing the AMHR2-ED binding molecules). In particular embodiments, the expression technology described in WO0202783 and WO0202738 to Bleck et al. (both of which are herein incorporated by reference in their entireties) is employed.

Mammalian host cells for expressing the recombinant antibodies of the invention include, for example, PER.C6™ cells (Crucell, The Netherlands), Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are generally produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present disclosure. For example, it may be desirable to transfect a host cell with DNA encoding either the light chain or the heavy chain of an antibody of this disclosure. Recombinant DNA technology may also be used to remove some or all of the DNA encoding either or both of the light and heavy chains that is not necessary for binding to AMHR2-ED. The molecules expressed from such truncated DNA molecules are also encompassed by the antibodies of the invention. In addition, bi-functional antibodies may be produced in which one heavy and one light chain are an antibody of the invention and the other heavy and light chain are specific for an antigen other than AMHR2-ED (e.g., by crosslinking an antibody of the invention to a second antibody by standard chemical crosslinking methods).

In certain embodiments, the antibodies and antibody fragments of the present invention are produced in transgenic animals. For example, transgenic sheep and cows may be engineered to produce the antibodies or antibody fragments in their milk (see, e.g., Pollock D P, et al., (1999) Transgenic milk as a method for the production of recombinant antibodies. J. Immunol. Methods 231:147-157, herein incorporated by reference). The antibodies and antibody fragments of the present invention may also be produced in plants (see, e.g., Larrick et al., (2001) Production of secretory IgA antibodies in plants. Biomol. Eng. 18:87-94, herein incorporated by reference). Additional methodologies and purification protocols are provided in Humphreys et al., (2001) Therapeutic antibody production technologies: molecules applications, expression and purification, Curr. Opin. Drug Discov. Devel. 4:172-185, herein incorporated by reference. In certain embodiments, the antibodies or antibody fragments of the present invention are produced by transgenic chickens (see, e.g., US Pat. Pub. Nos. 20020108132 and 20020028488, both of which are herein incorporated by reference).

In certain embodiments, the AMHR2-ED binding molecules of the present invention (e.g., as antibodies or antibody fragments) are useful for immunoassays which detect or quantify AMHR2-ED in a sample (e.g., a purified blood sample from a subject). In some embodiments, an immunoassay for AMHR2-ED typically comprises incubating a biological sample in the presence of a detectably labeled antibody or antibody fragment of the present invention capable of selectively binding to AMHR2-ED, and detecting the labeled peptide or antibody which is bound in a sample. Various clinical assay procedures are well known in the art.

The present disclosure provides immunoassay methods for determining the presence, amount or concentration of AMHR2-ED in a test sample. Any suitable assay known in the art can be used in such a method. Examples of such assays include, but are not limited to, immunoassay, such as sandwich immunoassay (e.g., monoclonal-polyclonal sandwich immunoassays, including radioisotope detection (radioimmunoassay (RIA)) and enzyme detection (enzyme immunoassay (EIA) or enzyme-linked immunosorbent assay (ELISA) (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), an ARCHITECT assay (ABBOTT), a bioluminescence resonance energy transfer (BRET), and homogeneous chemiluminescent assay, etc.

An AMHR2-ED binding molecule can be captured on beads or nitrocellulose, or on any other solid support which is capable of immobilizing soluble proteins (e.g., magnetic beads). An AMHR2-ED containing sample is then added to the support which is subsequently washed with suitable buffers to remove unbound proteins. A second, detectably labeled, molecule (e.g., antibody or peptide) that can bind to the AMHR2-ED binding molecule is added to the solid phase support that can then be washed with the buffer a second time to remove unbound molecules. The amount of bound label on the solid support can then be detected by known methods.

Detectably labeling AMHR2-ED binding molecule can be accomplished by coupling to an enzyme for use in an enzyme immunoassay (EIA), or enzyme-linked immunosorbent assay (ELISA). The linked enzyme reacts with the exposed substrate to generate a chemical moiety which can be detected, for example, by spectrophotometric, fluorometric or by visual means. Enzymes which can be used to detectably label the AMHR2-ED binding molecules of the present invention include, but are not limited to, malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase.

In some embodiments of the present invention, AMHR2-ED which is detected by the above assays can be present in a biological sample. Any sample containing AMHR2-ED can be used. Preferably, the sample is a biological fluid such as, for example, blood, serum, lymph, urine, cerebrospinal fluid, amniotic fluid, synovial fluid, a tissue extract or homogenate, and the like. However, the invention is not limited to assays using only these samples, as it is possible for one of ordinary skill in the art to determine suitable conditions which allow the use of other samples.

In situ detection can be accomplished by removing a histological specimen from a patient, and providing the combination of labeled AMHR2-ED binding molecules of the present disclosure to such a specimen. The AMHR2-ED binding molecule is preferably provided by applying or by overlaying the labeled AMHR2-ED binding molecule to a biological sample. Through the use of such a procedure, it is possible to determine not only the presence of AMHR2-EDs, but also the distribution of AMHR2-ED in the examined tissue.

In certain embodiments, provided here are kits for the detection of AMHR2-ED that include an AMHR2-ED detection molecule. Such kits may include any of the immunodiagnostic reagents described herein and may further include instructions for the use of the immunodiagnostic reagents in immunoassays for determining the presence of AMHR2-ED in a test sample. The kits may also include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit. The kit can additionally include one or more other controls. One or more of the components of the kit can be lyophilized, in which case the kit can further comprise reagents suitable for the reconstitution of the lyophilized components.

The various components of the kit may be provided in suitable containers as necessary, e.g., a microtiter plate. The kit can further include containers for holding or storing a sample (e.g., a container or cartridge for a sample). Where appropriate, the kit optionally also can contain reaction vessels, mixing vessels, and other components that facilitate the preparation of reagents or the test sample. The kit can also include one or more instrument for assisting with obtaining a test sample, such as a syringe, pipette, forceps, measured spoon, or the like.

### EXAMPLES

The following examples are provided in order to demonstrate and further illustrate certain preferred embodiments and aspects of the present invention and are not to be construed as limiting the scope thereof.

### Example 1

### Characterization of the Chimeric 4D12G1 Mab and its Humanized Variants 5 and 6 mAbs

This example describes testing anti-AMHR-ED chimeric and humanized antibodies Variants 5 and 6.

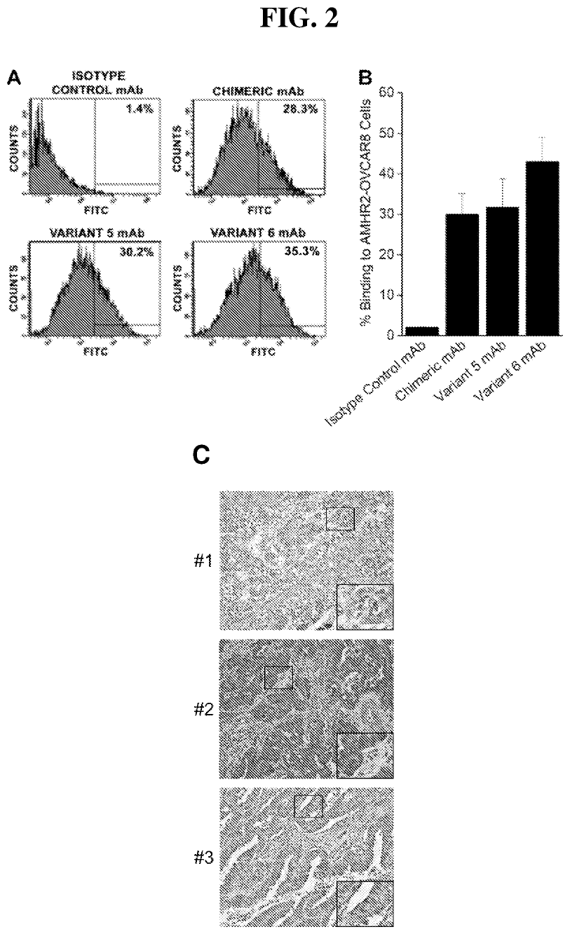

The human AMHR2-OVCAR8 cells expressing high levels of human AMHR2 were treated with Fc-receptor block (BD Biosciences, San Jose, CA) and immunostained with 5 μg of a human isotype control IgG1 mAb as well as with 5 μg of the chimeric 4D12G1 mAb and variants 5 and 6 of the humanized 4D12G1 mAb. Cells were then treated with FITC-labeled goat anti-human IgG (BD Biosciences) and analyzed by flow cytometry using a FACSAria II flow cytometer and BDFacsDiva software (BD Biosciences). FIG. 2 shows the percent binding of various mAbs to EOC Cells, showing that the Chimeric 4D12G1 mAb and its humanized variants 5 and 6 bind effectively to human EOC cells expressing AMHR2-ED. FIG. 2A shows immunostaining of the AMHR2-OVCAR8 cells with the humanized 4D12G1 mAb variants 5 and 6 compared favorably with the immunostaining obtained using the chimeric 4D12G1 mAb. FIG. 2B shows the staining comparison in column graph format. FIG. 2C demonstrates that Variant 6 can detect AMHR2-ED in tissue sections from primary HGSOCs by performing IHC. Insets show darkly staining regions of AMHR2-ED expression which occurs primarily in the parenchyma, with some staining also visible in the stroma.

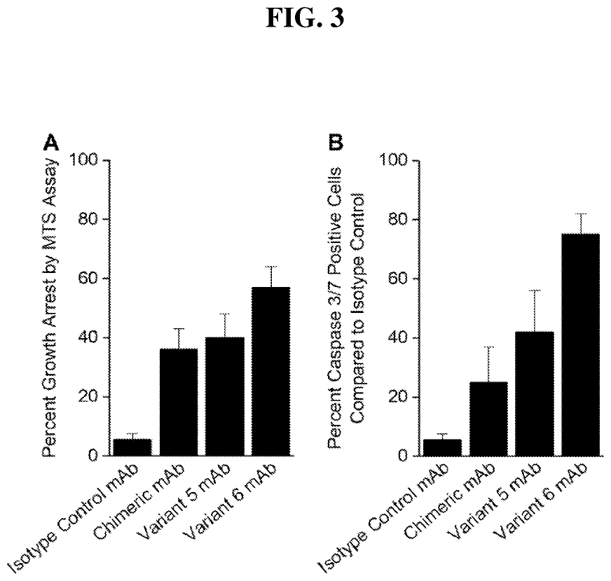

1×10⁴ AMHR2-OVCAR8 cells were incubated in microtiter wells with either 5 μg of affinity purified human isotype control IgG1 mAb, chimeric 4D12G1 mAb, or humanized 4D12G1 variant 5 or 6 mAbs. After 72 hours, the MTS assay was performed using the CellTiter 96 AQueous One Solution Cell Proliferation Assay (Promega, Madison, WI) according to the manufacturer's instructions. The bioreduction of the MTS tetrazolium compound by live cells into a soluble colored formazan product measured by absorbance at 490 nm is directly proportional to the number of living cells in culture. The percent of dead cells were compared to cultures treated with isotype control mAb. Next, AMHR2-OVCAR8 cells were incubated with the green fluorescent Caspase 3/7 dye IncuCyte (Essen BioScience, Ann Arbor, MI) for 16 hours in complement-free DMEM at 37° C. with 10 μg/ml of either isotype control mAb, chimeric 4D12G1 mAb, or humanized 4D12G1 variant 5 or 6 mAbs. Cell death was imaged in real-time using the IncuCyte S3 live-cell analysis (Essen BioScience), and the data were plotted as percent caspase-3 green fluorescent dye compared to cultures treated with isotype control mAb. FIG. 3 shows the results, showing that chimeric and humanized 4D12G1 mAbs induce programmed cell death (PCD). Specifically, FIG. 3 shows the chimeric 4D12G1 mAb and its humanized variants 5 and 6 induce PCD of Human EOC Cells Expressing AMHR2-ED.

AMHR2-OVCAR8 target cells were incubated at 2×10⁴ cells/microtiter well with human peripheral blood mononuclear cells (hPBMC; Lonza Group Ltd., Houston, TX) that contain the NK effector cells. Cells were cultured at a 10:1 hPBMC effector cell to AMHR2-OVCAR8 target cell ratio. Increasing concentrations of either affinity-purified human isotype control IgG1 mAb, chimeric 4D12G1 mAb, or humanized 4D12G1 variant 5 or 6 mAbs were added to these cultures. After 4 hours at 37° C., supernatant levels of lactate dehydrogenase (LDH) released from lysed target cells were measured using the CyQUANT LDH Assay kit (Thermo Fisher Scientific, Waltham, MA). The percent specific release of LDH from lysed AMHR2-OVCAR8 target cells was determined as a fraction of maximum release and plotted against the increasing concentrations of mAbs.

Next, human peripheral blood mononuclear cells (hPBMC) were obtained commercially (Lonza Group) and plated in RPMI-1640 onto non-coated microtiter wells at 37° C. to allow the monocytes to adhere. After one hour, wells were washed 3× with RPMI-1640 supplemented with 10% fetal bovine serum (Hyclone, Ogden, UT) to remove non-adherent cells. The remaining adherent monocytes were cultured in media containing 50 ng/ml recombinant human macrophage colony stimulating factor (ScienCell Research Laboratories, Carlsbad, CA) for 8 days with a change of media at day 3. The mature differentiated macrophages were detached by incubation on ice with cold PBS containing 5 mM EDTA for 10-15 minutes, counted, and labeled with 5-chloromethylfluorescein diacetate dye (CellTracker Green, Thermo Fisher). The macrophage effector cells and labeled target cells were added at a 10:1 effector to target cell ratio to microtiter wells preincubated at room temperature for 30 minutes with either isotype control mAb, chimeric mAb, Variant 5 mAb, or Variant 6 mAb at 100 ng/ml and 1,000 ng/ml. After 3 days, live target AMHR2-OVCAR8 cells were detected by flow cytometry.

The mouse 4D12G1 mAb recognized peptides spanning AMHR2-ED 11-32 (11EAPGVRGSTKTLGELLDTGTEL32; SEQ ID NO:33). Subsequent epitope mapping studies using peptides with alanine substitutions at each amino across the AMHR2-ED 11-32 sequence showed that alanine substituted peptides spanning AMHR2-ED 20-26 (20KTLGELL26; SEQ ID NO:34) were capable of dramatically inhibiting binding of the 4D12G1 mAb to AMHR2-ED in competitive ELISAs. The importance of this binding domain for the mouse 4D12G1 mAb lies in the fact that the 20KTLGELL26 of AMHR2-ED is juxtaposed to AMHR2-ED 4-15 (4RRTCVFFEAPGV15; SEQ ID NO:35), the primary binding site for the native ligand, anti-Müllerian hormone (AMH). In addition, the 20KTLGELL26 sequence of AMHR2-ED is adjacent to AMHR2-ED 34-42 (34RAIRCLYSR42; SEQ ID NO:36), the secondary binding site for AMH. The binding closeness of the 4D12G1 mAb to the primary and secondary binding sites for AMH explains the ability of the 4D12G1 mAb to compete with the AMH native ligand for binding to AMHR2-ED. Moreover, the close proximity of the 4D12G1 binding site to the key primary and secondary AMH binding sites provides a substantial advantage for consistently signaling programmed cell death following binding of the 4D12G1 mAb to the AMHR2-ED receptor on EOC cells. Thus, it is important to determine that the humanized 4D12G1 variant 5 and 6 mAbs recognize the same AMHR2-ED 20-26 (20KTLGELL26) sequence recognized by the original mouse 4D12G1 mAb.

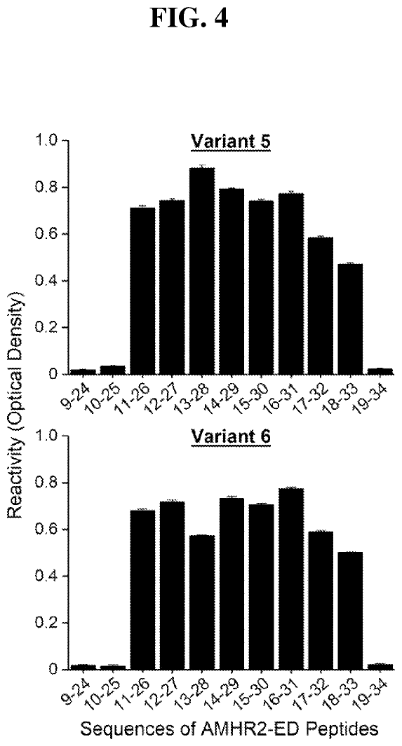

To this end we tested the binding of the humanized 4D12G1 variants 5 and 6 mAbs to an overlapping 16-mer peptide series spanning the human AMHR2-ED 9-34 sequence and found that both variant mAbs recognize the important AMHR2-ED 20-26 (20KTLGELL26) sequence (FIG. 4). We used an overlapping set of 16-mer peptides spanning the AMHR2-ED 9-34 amino acids that contain the 20KTLGELL26 sequence known to be the critical binding region of AMHR2-ED recognized by the native mouse 4D12G1 mAb. Each 16-mer peptide was shifted by one amino acid and all cysteine residues were replaced by serine to avoid disulfide bond formation (Thermo Fisher, Waltham, MA). Individual 16-mer peptides were used to coat the flat-bottom wells in 96-well MaxiSorp microtiter plates (Thermo Fisher) for 24 hours at 4° C. Wells were washed the following day with PBS followed by blocking with 1% BSA in PBS for 1 hour at room temperature. Wells were then washed with PBS containing 0.02% Tween-20 and incubated with the humanized 4D12G1 variant 5 and 6 mAbs at 5 μg/ml) in liquid phase for 24 hours at 4° C. Wells were then washed with PBS containing 0.02% Tween-20 and incubated with a secondary anti-mouse IgG conjugated with horseradish peroxidase (HRP; MilliporeSigma, Burlington, MA) for 1 hour at room temperature, followed by the addition of 2,2′-Azino-bis(3-ethylbenzothiazoline-6-sulfonic acid) diammonium salt (ABTS) substrate (Sigma-Aldrich). Optical densities of the color reactions were determined by absorbance at 405 nm. Our results indicate that the humanization process did not alter epitope recognition of the humanized 4D12G1 variant 5 mAb (upper panel) and the humanized 4D12G1 variant 6 mAb (lower panel) when compared to the binding observed using the native mouse 4D12G1 mAb [6]. FIG. 4 shows epitope mapping of the humanized 4D12G1 variant 5 and 6 mAbs in binding ELISAs to AMHR2-ED peptides.

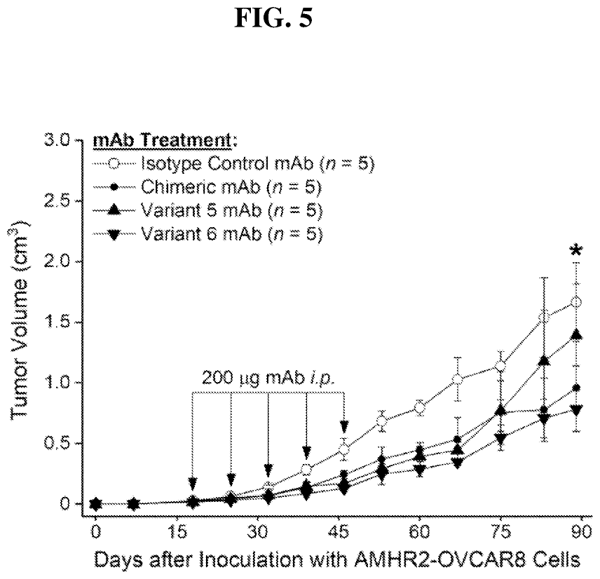

Next, immunodeficient female NOD-scid IL2Rgammanull (NSG) mice were purchased at 6-8 weeks of age from the Cleveland Clinic Biological Resources Unit (Cleveland, OH). 5×106 AMHR2-OVCAR8 cells were inoculated subcutaneously into the flanks of each NSG mouse. Tumor growth was measured weekly using a Vernier caliper. When tumors became palpable, mice were separated into 4 groups of 5 mice each for intraperitoneal injection of 200 μg of mAb weekly for 5 continuous weeks. Experiments were terminated when tumor length reached 17 mm in any direction. Tumor volume (cm³) was plotted against the days following AMHR2-OVCAR8 inoculation. All protocols for animal research met with the prior approval of the Cleveland Clinic's Institutional Animal Care and Use Committee (IACUC #2018-2090) in compliance with the Public Health Service policy on humane care and use of research animals. FIG. 5 show the results, showing in vivo inhibition of human EOC growth by humanized mAbs. Specifically, FIG. 5 shows the 4D12G1 variant 6 mAb, but not the 4D12G1 variant 5 mAb, inhibited the growth of human AMHR2-OVCAR8 EOCs inoculated into immunodeficient NSG mice.

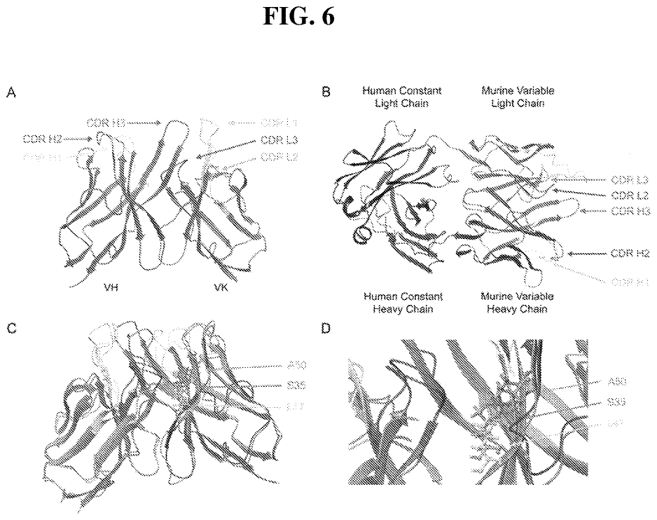

The CDRs of the variable heavy and light chains of mouse 4D12G1 were determined for the humanization process (FIG. 6a). For ch4D12G1, the human constant (heavy and light chains) domain was spliced with the murine variable (heavy and light chains) domain by replacing the mouse constant domain of the parental 4D12G1 (FIG. 6b). The Hu4D12G1-6 antibody variant has ˜90% homology to the Fab region of mouse 4D12G1 mAb (FIGS. 1c and 1d). The key amino acid residues of the parental 4D12G1 Fab fragment, critical for 4D12G1 antibody expression and binding to AMHR2-ED, were determined using the BioLuminate Protein Structure Alignment algorithm. Using site-directed mutagenesis, it was found that three amino acid residues Ala50 (alanine after CDR2), Ser35 (serine after CDR1), and Leu47 (leucine at the interface position) within the variable heavy chain of the antibody are critical for binding to AMHR2-ED. The biologic significance of these three residues is supported by their location near the CDR loops at the interface of the heavy and light chains in the 3D model. Importantly, these amino acid residues were unaltered in the generation of the Hu4D12G1-6 humanized 4D12G1 variant (FIGS. 6c and 6d).

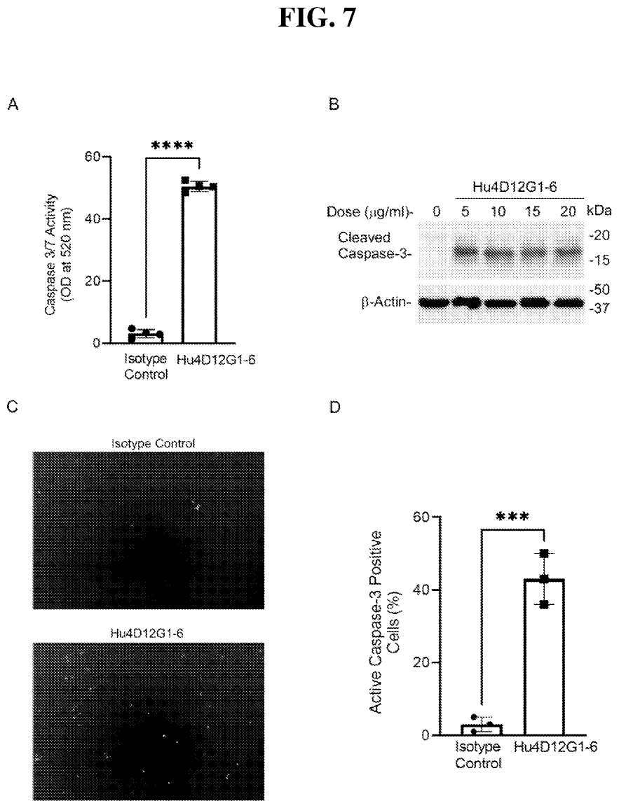

To test the ability of Hu4D12G1-6 to induce cell death, we performed a CaspaTag Caspase-3/7 assay following treatment of AMHR2-OVCAR8 cells with 25 μg/ml antibodies for 48 hours compared to treatment with isotype control. The results showed that Hu4D12G1-6 induced significantly high Caspase-3/7 activity in AMHR2-OVCAR8 cells than the isotype control (FIG. 7a, P<0.0001). Caspase-3 is a 37 kDa protein, and its cleavage into fragment(s) of 17 and/or 19 kDa is the hallmark of apoptotic cell death [24]. Thus, we next examined whether Hu4D12G1-6 is able to cleave Caspase-3 indicating induction of apoptosis. AMHR2-OVCAR8 cells were treated with different concentrations of Hu4D12G1-6 for 16 hours, and cell lysates were examined on Western blots for the presence of cleaved Caspase-3. Immunostaining with antibody to the 42 kDa protein β-actin (Sigma-Aldrich) was used to confirm uniform lysate loading. The results indicate that Hu4D12G1-6 treatment with doses as low as 5 g/ml caused the appearance of cleaved fragments of 17 kDa Caspase-3 in cell lysates (FIG. 7b). Finally, we tested whether Hu4D12G1-6 can facilitate direct cell death, as assessed by Incucyte real-time live imaging of active Caspase-3/7 positive green fluorescent AMHR2-OVCAR8 cells treated in vitro for 16 hours with 10 μg/ml mAbs in DMEM supplemented with heat-inactivated serum (FIG. 7c). The results indicate that treatment with Hu4D12G1-6 induced a significant level of apoptosis (˜40%) in AMHR2-OVCAR8 cells compared to the isotype control (˜3%) (FIG. 7d; P=0.0007).

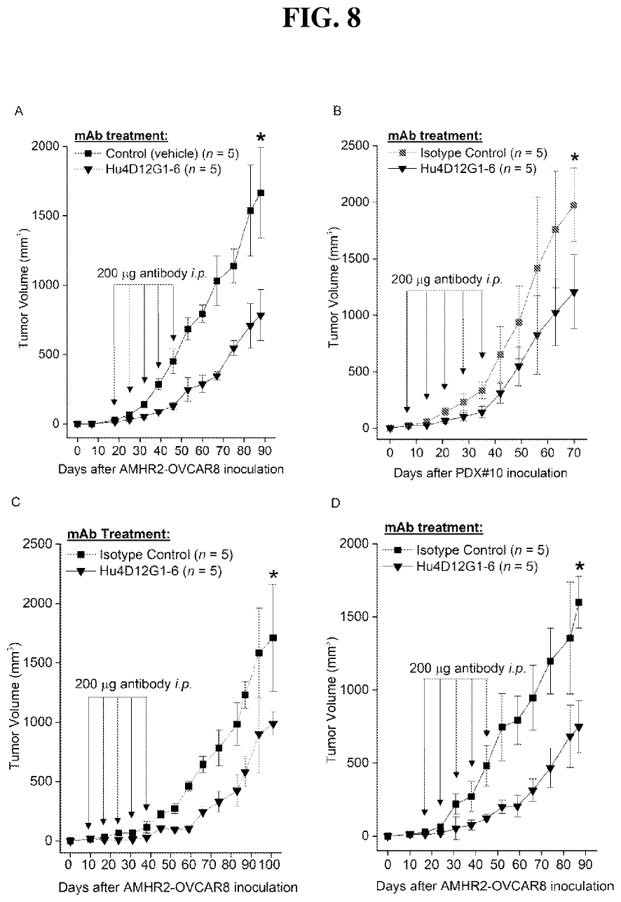

To evaluate the efficacy of Hu4D12G1-6 in vivo, human AMHR2-OVCAR8 cells or human HGSOC-derived PDX #10 tumor cells were injected s.c. to the flanks of immunodeficient NSG mice. When tumors became palpable, mice were injected i.p. with 200 μg of either Hu4D12G1-6 or an isotype control once weekly for 5 consecutive weeks. The results show that treatment with Hu4D12G1-6 significantly inhibited the growth of AMHR2-OVCAR8 (FIG. 8a; P<0.0001) and PDX #10 tumors (FIG. 8b; P=0.0005) in severely immunodeficient NSG mice. Similar results were obtained in additional independent experiments by administration of Hu4D12G1-6 in two different human EOC xenograft models. To assess the immune effector functions, including ADCC by Hu4D12G1-6 in vivo, we used humanized NSG-IL-15 (huNSG-IL-15) mice xenografted with AMHR2-OVCAR8 cells. The results showed significant inhibition of tumor (FIG. 8c; P<0.0001) to a similar degree observed in NSG mice (FIG. 8a). Next, to assess another immune effector function, ADCP, by our antibody in vivo, we inoculated AMHR2-OVCAR8 cells in humanized NSG-SGM3 (huNSG-SGM3) mice. Again, the results showed significant inhibition of tumor (FIG. 8d; P<0.0001) to a similar degree observed in NSG mice (FIG. 8a). Although the data from the huNSG-IL-15 and the huNSG-SGM3 models demonstrate significant anti-tumor effects, these outcomes were not enhanced by ADCC or ADCP, respectively. Taken together, our results suggest that Hu4D12G1-6 is highly effective in inhibiting human EOCs in preclinical murine models primarily by apoptosis, without the significant contribution of ADCC or ADCP.

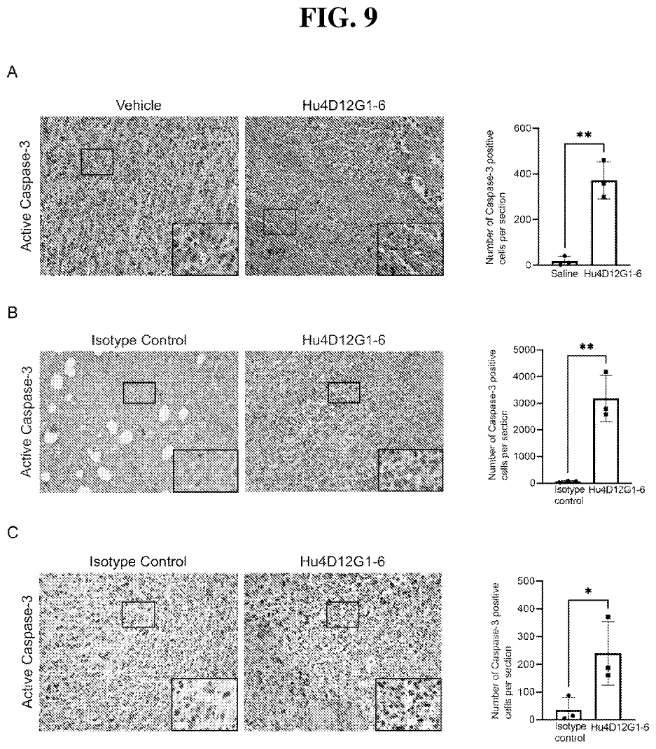

We further investigated the mechanism of tumor inhibition by Hu4D12G1-6 treatment in vivo. We found that programmed cell death is the primary mechanism of EOC cell death in Hu4D12G1-6 treatment in immunodeficient NSG mice, as demonstrated by active Caspase-3 (FIG. 9a) of tumor sections. These results are similar to our prior studies with mouse 4D12G1 mAb [20]. Next, we evaluated the PDX #10 tumor bed following antibody treatment and found that cells with active Caspase-3 are predominantly present in the Hu4D12G1-6 treated tumor environment compared with the isotype control (FIG. 9b). Finally, we characterized the EOC tumor bed of huNSG-SGM3 mice by performing active Caspase-3 IHC and demonstrated that apoptotic cell death is strongly evident in tumor sections from mice treated with Hu4D12G1-6, but not with those treated with isotype control (FIG. 9c). The Caspase-3-positive cells per section from three independent sections of each treatment were quantified (FIG. 9a-c; right side). These results indicate that the number of cells positively stained for Caspase-3 after Hu4D12G1-6 treatment is significant compared with the control (11a, P=0.0019; 11b, P=0.0034; 11c, P=0.0451). Collectively, these data indicate that Hu4D12G1-6 treatment mediates its effect in the EOC tumor bed primarily by inducing apoptosis.

### REFERENCES

1. Masiakos et al., Clin Cancer Res. 1999; 5:3488-3499.
2. Bakkum-Gamez et al., Gynecol Oncol. 2008; 108:141-148.
3. Song et al., Int J Oncol. 2009; 34:1583-1591.
4. Sakalar et al., J Immunol Res. 2015; 2015:630287.
5. Mazumder et al., Cancer Prev Res (Phila). 2017; 10:612-624.
6. Mazumder et al., Oncotarget. 2020; 11:1894-1910.
7. Rodriguez-Garcia et al., Mol Ther. 2020; 28:548-560.
8. Shoemaker et al., Cancer Prevention Research (Philadelphia). 2017 November; 10 (11): 607-608.
9. Elia Ben-Ari (NCI Staff Writer). Experimental Ovarian Cancer Vaccine Shows Promise in Mice, Cancer Currents Blog; Nov. 17, 2017 (https://www.cancer.gov/news-events/cancer-currents-blog/2017/ovarian-cancer-vaccine)

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, medicine, and molecular biology or related fields are intended to be within the scope of the following claims.

We claim:

1. A composition comprising an AMHR2-ED binding molecule, wherein said AMHR2-ED binding molecule comprises:
a) a heavy chain variable region, wherein said heavy chain variable region comprises:
i) a CDRH1 amino acid sequence comprising SEQ ID NO:13, or SEQ ID NO: 13 one with one or two conservative amino acid changes,
ii) a CDRH2 amino acid sequence comprising SEQ ID NO:14, and
iii) a CDRH3 amino acid sequence comprising SEQ ID NO:15, or SEQ ID NO: 15 with one or two conservative amino acid changes, and optionally
b) a light chain variable region, wherein said light chain variable region comprises;
i) a CDRL1 amino acid sequence comprising SEQ ID NO:18, or SEQ ID NO: 18 with one or two conservative amino acid changes,
ii) a CDRL2 amino acid sequence comprising SEQ ID NO:19, or SEQ ID NO: 19 with one or two conservative amino acid changes, and
iii) a CDRL3 amino acid sequence comprising SEQ ID NO:20, or SEQ ID NO: 20 with one or two conservative amino acid changes.

2. The composition of claim 1, wherein said AMHR2-ED binding molecule is an antibody, minibody, diabody, scFv, or antibody fragment capable of binding human AMHR2-ED.

3. The composition of claim 2, wherein said antibody fragment is an Fab, or Fv antibody fragment.

4. The composition of claim 2, wherein said antibody or antibody fragment comprises an antigen binding portion of the 4D12G1 Variant 5 antibody, or 4D12G1 Variant 6 antibody.

5. The composition of claim 1, wherein said heavy chain and/or light chain variable region comprises a human framework region.

6. The composition of claim 1, wherein said AMHR2-ED binding molecule further comprises a light chain constant region and a CH1 heavy chain constant region.

7. The composition of claim 6, wherein said AMHR2-ED binding molecule further comprises a CH2 heavy chain constant region and/or a CH3 heavy chain constant region.

8. The composition of claim 7, wherein said light chain constant region is human or a humanized murine, and/or wherein said CH1, CH2, and CH3 heavy chain constant regions are human or are humanized murine.

9. The composition of claim 1, wherein said AMHR2-ED binding molecule is an antibody or antigen binding portion thereof that has an Fc region characterized in that it: i) has an Fc cellular binding site; ii) has a Fc complement binding site; and/or 3) has a linked toxin that is internalized into a cell.

10. The composition of claim 1, wherein said AMHR2-ED binding molecule comprises an antibody, wherein the light chain constant region of said antibody is selected from: IgG Kappa and IgG Lambda, and wherein the heavy chain constant region of said antibody is selected from: IgG1, IgG2, IgG3, and IgG4.

11. The composition of claim 1, wherein said AMHR2-ED binding molecule comprises an antibody, or antigen binding portion thereof, which is glycosylated or non-glycosylated.

12. The composition of claim 1, further comprising a physiologically tolerable buffer.

13. The composition of claim 1, wherein said heavy chain variable regions comprises SEQ ID NO:12 or 22, or SEQ ID NOs: 12 or 22 with one or more conservative amino acid changes.

14. The composition of claim 1, wherein said light chain variable region comprises SEQ ID NO: 17 or 27, or SEQ ID NOs: 17 or 27 with one or more conservative amino acid changes.

15. A composition comprising at least one of the following:
a) a first nucleic acid sequence encoding a heavy chain variable region, wherein said heavy chain variable region comprises:
i) a CDRH1 amino acid sequence comprising SEQ ID NO:13, or SEQ ID NO: 13 one with one or two conservative amino acid changes,
ii) a CDRH2 amino acid sequence comprising SEQ ID NO:14, and
iii) a CDRH3 amino acid sequence comprising SEQ ID NO:15, or SEQ ID NO: 15 with one or two conservative amino acid changes, and optionally
b) a second nucleic acid sequence encoding a light chain variable region, wherein said light chain variable region comprises:
i) a CDRL1 amino acid sequence comprising SEQ ID NO:18, or SEQ ID NO: 18 with one or two conservative amino acid changes,
ii) a CDRL2 amino acid sequence comprising SEQ ID NO:19, or SEQ ID NO: 19 with one or two conservative amino acid changes, and
iii) a CDRL3 amino acid sequence comprising SEQ ID NO:20, or SEQ ID NO: 20 with one or two conservative amino acid changes.

16. The composition of claim 15, further comprising an expression vector, and wherein said first and/or second nucleic acid sequences are present in said expression vector.

17. The composition of claim 15, wherein said light and heavy chain variable regions are part of an AMHR2-ED binding molecule.

18. The composition of claim 17, wherein said AMHR2-ED binding molecule is an antibody, minibody, diabody, scFv, or antibody fragment capable of binding human AMHR2-ED.

19. The composition of claim 18, wherein said antibody fragment is a Fab or Fv antibody fragment.

20. The composition of claim 18, wherein said antibody or antibody fragment comprises an antigen binding portion of the 4D12G1 Variant 5 antibody, or 4D12G1 Variant 6 antibody.

21. The composition of claim 15, wherein said light and/or heavy chain variable region comprises a human framework region.

22. The composition of claim 17, wherein said AMHR2-ED binding molecule further comprises a light chain constant region and a CH1 heavy chain constant region.

23. The composition of claim 22, wherein said AMHR2-ED binding molecule further comprises a CH2 heavy chain constant region.

24. The composition of claim 23, wherein said AMHR2-ED binding molecule further comprises a CH3 heavy chain constant region.

25. The composition of claim 24, wherein said light chain constant region is human or humanized murine, and/or wherein said CH1, CH2, and CH3 heavy chain constant regions are human or are humanized murine.

26. The composition of claim 17, wherein said AMHR2-ED binding molecule comprises an antibody, wherein the light chain constant region of said antibody is selected from: IgG Kappa and IgG Lambda, and wherein the heavy chain constant region of said antibody is selected from: IgG1, IgG2, IgG3, and IgG4.

27. The composition of claim 15, wherein said heavy chain variable regions comprises SEQ ID NO:12 or 22, or SEQ ID NOs: 12, or 22 with one or more conservative amino acid changes.

28. The composition of claim 15, wherein said light chain variable region comprises SEQ ID NO: 17, 27, or SEQ ID NOs: 17, or 27 with one or more conservative amino acid changes.

29. A method of treating or preventing ovarian and/or endometrial cancer comprising:
treating a subject with an AMHR2-ED binding molecule as recited in any of claims 1-14, or an expression vector encoding said AMHR2-ED binding molecule, and
wherein said subject has, or is suspected to develop, ovarian and/or endometrial cancer.

30. The method of claim 29, wherein said ovarian and/or endometrial cancer is ovarian cancer.

31. The method of claim 29, wherein said ovarian and/or endometrial cancer is endometrial cancer.

32. The method of claim 29, wherein said AMHR2-ED binding molecule is an antibody or antigen binding portion thereof.

33. The method of claim 32, wherein said antibody or antigen binding portion thereof is a human antibody or antigen binding portion thereof.

34. The method of claim 32, wherein said antibody or antigen binding portion thereof is a humanized antibody or antigen binding portion thereof.

35. The method of claim 29, wherein said heavy chain variable regions comprises SEQ ID NO: 12 or 22, or SEQ ID NOs: 12, or 22 with one or more conservative amino acid changes.

36. The method of claim 29, wherein said light chain variable region comprises SEQ ID NO: 17 or 27, or SEQ ID NOs: 17 or 27 with one or more conservative amino acid changes.

37. A method of detecting AMHR2-ED in a sample comprising:
a) contacting a sample with the AMHR2-ED binding molecule of any of claims 1-14,
wherein said sample is suspected of containing AMHR2-ED, and wherein said AMHR2-ED binding molecule forms a complex with said AMHR2-ED if present in said sample; and
b) detecting the presence or absence of said complex in said sample.

38. The method of claim 37, wherein said sample is from a subject that has, or is suspected to develop, ovarian and/or endometrial cancer.

39. The method of claim 37, wherein said AMHR2-ED binding molecule comprises a detectable label.

40. The method of claim 37, further comprising contacting said sample with a conjugate molecule capable of binding to said AMHR2-ED binding molecule, wherein said conjugate molecule comprises a detectable label.
